(19) United States
(12) Patent Application Publication
(10) Pub. No.: US 2025/0114796 A1
(43) Pub. Date: Apr. 10, 2025

(54) Grinding Aid Compositions and Methods of Use

(71) Applicant: Locus Solutions IPCo, LLC, Solon, OH (US)

(72) Inventor: Gabriela KNESEL, Solon, OH (US)

(21) Appl. No.: 18/698,652

(22) PCT Filed: Jun. 22, 2023
(86) PCT No.: PCT/US2023/068861

§ 371 Date: Apr. 4, 2024

Related U.S. Application Data
(60) Provisional application No. 63/356,553, filed on Jun. 29, 2022.

(51) Int. Cl. B02C 23/06; C04B 7/52; C12N 1/16

(52) U.S. Cl. CPC B02C 23/06; C04B 7/52; C12N 1/16

(57) ABSTRACT

The subject invention provides safe, environmentally-friendly compositions and efficient methods for grinding materials. More specifically, the subject invention provides compositions derived from microorganisms for grinding, which can be used for increasing the rate of grinding and/or the amount of ground particles.

### CROSS-REFERENCE TO RELATED APPLICATION

This application claims priority to U.S. Provisional Patent Application No. 63/356,553, filed Jun. 29, 2022, which is incorporated herein by reference in its entirety.

### BACKGROUND OF THE INVENTION

Efficiently removing deposits from the earth associated with various mining and quarrying practices commonly employs the use of grinding aids. During mining, grinding aids can be added to improve the grinding efficiency of a mined ore or mineral by decreasing the amount of energy required to create new surfaces during grinding.

Depending on the type of grinding device being used, including, for example, dry or wet grinding, and the type of grinding mill, different chemical substances are used to aid the process.

In dry grinding systems, organic chemicals, such as alcohols, glycols, phenols, ketones, acids, and hydrocarbons, are used. The chemicals can alter the specific surface energy and the agglomeration tendencies of the ore and minerals being ground as well as the transfer of kinetic energy from the grinding medium to the minerals.

The principal commodities that employ grinding aids are cement and limestone. For cement, a low-value commodity, grinding accounts for a major portion of production costs. In metallic minerals and most other hard industrial minerals, grinding is a relatively small-cost item; therefore, economizing measures are employed in other, more expensive steps of the process.

Wet grinding aids are used in the ball milling of mineral ores. The process is increasingly used to enable better separation of particles containing the desired end product, and to save chemicals used in later process steps. Nickel, cobalt, iron ore, and bauxite are minerals that use wet grinding aids in their production.

Currently, the use of grinding aid has largely been limited to large-tonnage grinding methods, including, for example, tube mills, rod mills, ball mills, and autogenous and semi-autogenous grinding. Additionally, grinding aids are normally manufactured from specialty chemicals derived from petrochemicals.

Therefore, novel, improved grinding aid compositions and methods are needed.

### BRIEF SUMMARY OF THE INVENTION

The subject invention relates generally to grinding aid compositions and methods of using these compositions. More specifically, the subject invention provides environmentally-friendly grinding aid compositions and methods for grinding, such as, for example, during the processing of mined ores and minerals. In certain embodiments, existing methods can incorporate the subject compositions and methods.

Advantageously, the compositions and methods of the subject invention increase the efficiency of grinding and can decrease the chemical usage, including chemical surfactant usage, required for grinding. Accordingly, the subject invention can be useful for reducing the time needed for mining or production of various products, including, for example, Portland cement.

In certain embodiments, the subject invention provides compositions comprising components that are derived from microorganisms. In certain embodiments, the composition comprises a microbial biosurfactant. In certain embodiments, the composition comprises one or more biosurfactants, and, optionally, other compounds, such as, for example, water, chemical surfactants, acrylic polymer dispersants, rheology modifiers, polymers, alcohols, glycols, phenols, lignosulfates, ketones, acids, hydrocarbons, fatty acids, fatty acid salts, or any combination thereof.

In certain embodiments, the biosurfactant of the composition is utilized in crude form. The crude form can comprise, in addition to the biosurfactant, fermentation broth in which a biosurfactant-producing microorganism was cultivated, residual microbial cell matter or live or inactive microbial cells, residual nutrients, and/or other microbial growth by-products.

In some embodiments, the biosurfactant is utilized after being extracted from a fermentation broth and, optionally, purified.

The biosurfactant according to the subject invention can be a glycolipid (e.g., sophorolipids, rhamnolipids, cellobiose lipids, mannosylerythritol lipids and trehalose lipids), lipopeptide (e.g., surfactin, iturin, fengycin, arthrofactin, and lichenysin), flavolipid, phospholipid (e.g., cardiolipins), fatty acid ester compound, fatty acid ether compound, and/or high molecular weight polymers such as lipoproteins, lipopolysaccharide-protein complexes, and polysaccharide-protein-fatty acid complexes.

In certain specific embodiments, the biosurfactant is a sophorolipid (SLP), including linear SLP, lactonic SLP, acetylated SLP, de-acetylated SLP, salt-form SLP, esterified SLP derivatives, amino acid-SLP conjugates, and other SLP derivatives or isomers that exist in nature and/or are produced synthetically. In preferred embodiments, the SLP is a linear SLP or a derivatized linear SLP.

In certain embodiments, the subject invention provides a method for grinding, wherein the method comprises the following step: contacting a grinding aid composition according to the subject invention to a mineral, ore, element, or other material.

In some embodiments, the method enhances the homogeneity of ground particles or increases the rate of grinding and/or the amount of ground particles in a distinct time period. In certain embodiments, the resulting ground particles can be less than about 10 cm, about 1 cm, about 1 mm, about 500 μm, about 100 μm, about 10 μm, about 1 μm, about 100 nm, about 10 nm, or about 1 nm in diameter.

In some embodiments, the method comprises contacting a grinding aid composition comprising a biosurfactant and, optionally, other components, such as, for example, water, chemical surfactants, acrylic polymer dispersants, rheology modifiers, polymers, alcohols, glycols, phenols, lignosulfates, ketones, acids, hydrocarbons, fatty acids, fatty acid salts, to a mineral, ore, element, or other material. The mineral, ore, element, or other material can be a liquid or solid. In certain embodiments, the grinding aid composition can be applied to the mineral, ore, element, or other material for a period of time and/or until a distinct volume of the composition has been applied. The step can be repeated as many times as necessary to achieve a desired amount of ground particles or rate of grinding of the mineral, ore, element, or other material.

In certain embodiments, the grinding aid composition according to the subject invention is effective due to decreasing the specific surface energy of particles required to make a new surface in a liquid or solid containing a mineral, ore, element, or other material. For example, in some embodiments, a sophorolipid will neutralize the particles or weaken the grain boundary planes.

In certain embodiments, the methods of the subject invention result in at least a 25% increase in rate of grinding of solids, preferably at least a 50% increase, after one treatment. In certain embodiments, the mineral, ore, element, or other material can be treated multiple times to further increase the amount of ground particles or rate of grinding.

Advantageously, in certain embodiments, the grinding aid composition according to the subject invention can be effective and efficient at creating a ground particle. Furthermore, the methods of the subject invention do not require complicated equipment or high energy consumption, and production of the composition can be performed on site, for example, at a mine or industrial production facility.

### DETAILED DESCRIPTION

The subject invention relates generally to the grinding of particles. More specifically, the subject invention provides environmentally-friendly compositions and methods for grinding solids or solid particles contained within liquids derived from mined ores, elements, minerals, or other materials. Accordingly, the subject invention is useful for improving the efficiency and efficacy of methods of grinding. Advantageously, the compositions and methods of the subject invention permit an increase in the grinding rate of particles using safe, environmentally-friendly compositions.

### Selected Definitions

As used herein, “applying” a composition or product refers to contacting it with a target or site such that the composition or product can have an effect on that target or site. The effect can be due to, for example, microbial growth and/or the action of a biosurfactant or other microbial growth by-product.

As used herein, a “biofilm” is a complex aggregate of microorganisms, such as bacteria, yeast, or fungi, wherein the cells adhere to each other and/or to a surface using an extracellular matrix. The cells in biofilms are physiologically distinct from planktonic cells of the same organism, which are single cells that can float or swim in liquid medium.

As used herein, an “isolated” or “purified” nucleic acid molecule, polynucleotide, polypeptide, protein or organic compound such as a small molecule (e.g., those described below), is substantially free of other compounds, such as cellular material, with which it is associated in nature. A purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state. An isolated microbial strain means that the strain is removed from the environment in which it exists in nature. Thus, the isolated strain may exist as, for example, a biologically pure culture, or as spores (or other forms of the strain) in association with a carrier.

In certain embodiments, purified compounds are at least 60% by weight the compound of interest. Preferably, the preparation is at least 75%, more preferably at least 90%, and most preferably at least 98%, by weight the compound of interest. For example, a purified compound is one that is at least 80%, 85%, 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

A “metabolite” refers to any substance produced by metabolism or a substance necessary for taking part in a particular metabolic process. A metabolite can be an organic compound that is a starting material, an intermediate in, or an end product of metabolism. Examples of metabolites include, but are not limited to, enzymes, acids, solvents, alcohols, proteins, vitamins, minerals, microelements, amino acids, biopolymers and biosurfactants.

Ranges provided herein are understood to be shorthand for all of the values within the range. For example, a range of 1 to 20 is understood to include any number, combination of numbers, or sub-range from the group consisting 1, 2, 3, 4, 5, 6, 7, 8, 9, 10, 11, 12, 13, 14, 15, 16, 17, 18, 19 and 20, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein a “reduction” or “decrease” means a negative alteration, and an “increase” means a positive alteration, wherein the negative or positive alteration is at least 0.001%, 0.01%, 0.1%, 0.5%, 1%, 5%, 10%, 15%, 20%, 25%, 30%, 35%, 40%, 45%, 50%, 55%, 60%, 65%, 70%, 75%, 80%, 85%, 90%, 95% or 100%.

As used herein, “surfactant” means a compound that lowers the surface tension (or interfacial tension) between two liquids or between a liquid and a solid. Surfactants act as, e.g., detergents, wetting agents, emulsifiers, foaming agents, and/or dispersants. A “biosurfactant” is a surface-active substance produced by a living cell and/or using naturally-derived substrates.

Biosurfactants are a structurally diverse group of surface-active substances consisting of two parts: a polar (hydrophilic) moiety and non-polar (hydrophobic) group. Due to their amphiphilic structure, biosurfactants can, for example, increase the surface area of hydrophobic water-insoluble substances, increase the water bioavailability of such substances, and change the properties of bacterial cell surfaces. Biosurfactants can also reduce the interfacial tension between water and oil and, therefore, lower the hydrostatic pressure required to move entrapped liquid to overcome the capillary effect. Biosurfactants accumulate at interfaces, thus reducing interfacial tension and leading to the formation of aggregated micellar structures in solution. The formation of micelles provides a physical mechanism to mobilize, for example, oil in a moving aqueous phase.

The ability of biosurfactants to reduce the surface tension also permits their use as antibacterial, antifungal, and hemolytic agents to, for example, control pests and/or microbial growth.

Typically, the hydrophilic group of a biosurfactant is a sugar (e.g., a mono-, di-, or polysaccharide) or a peptide, while the hydrophobic group is typically a fatty acid. Thus, there are countless potential variations of biosurfactant molecules based on, for example, type of sugar, number of sugars, size of peptides, which amino acids are present in the peptides, fatty acid length, saturation of fatty acids, additional acetylation, additional functional groups, esterification, polarity and charge of the molecule.

These variations lead to a group of molecules comprising a wide variety of classes, including, for example, glycolipids (e.g., sophorolipids, rhamnolipids, cellobiose lipids, mannosylerythritol lipids and trehalose lipids), lipopeptides (e.g., surfactin, iturin, fengycin, arthrofactin and lichenysin), flavolipids, phospholipids (e.g., cardiolipins), fatty acid ester compounds, and high molecular weight polymers such as lipoproteins, lipopolysaccharide-protein complexes, and polysaccharide-protein-fatty acid complexes. Each type of biosurfactant within each class can further comprise subtypes having further modified structures.

Like chemical surfactants, each biosurfactant molecule has its own HLB value depending on its structure; however, unlike production of chemical surfactants, which results in a single molecule with a single HLB value or range, one cycle of biosurfactant production typically results in a mixture of biosurfactant molecules (e.g., subtypes and isomers thereof).

The phrases “biosurfactant” and “biosurfactant molecule” include all forms, analogs, orthologs, isomers, and natural and/or anthropogenic modifications of any biosurfactant class (e.g., glycolipid) and/or subtype thereof (e.g., sophorolipid).

As used herein, the term “sophorolipid,” “sophorolipid molecule,” “SLP” or “SLP molecule” includes all forms, and isomers thereof, of SLP molecules, including, for example, acidic (linear) SLP (ASL) and lactonic SLP (LSL). Further included are mono-acetylated SLP, di-acetylated SLP, esterified SLP, SLP with varying hydrophobic chain lengths, cationic and/or anionic SLP with fatty acid-amino acid complexes attached, esterified SLP, SLP-metal complexes, SLP-salt derivatives (e.g., a sodium salt of a linear SLP), and other, including those that are and/or are not described within in this disclosure.

In preferred embodiments, the SLP molecules according to the subject invention are represented by General Formula (1) and/or General Formula (2), and include 30 or more compounds having different fatty acid chain lengths (R³), and, in some instances, having an acetylation or protonation at R¹ and/or R².

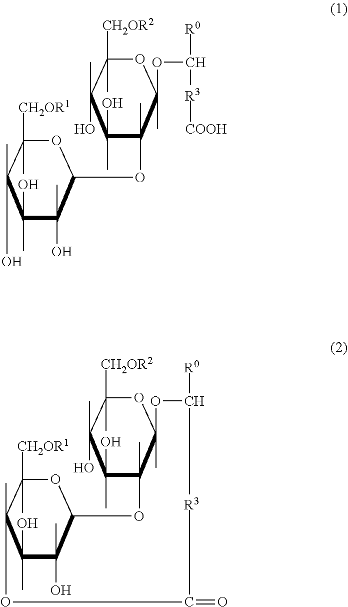

In General Formula (1) or (2), R⁰ can be either a hydrogen atom or a methyl group. R¹ and R² are each independently a hydrogen atom or an acetyl group. R³ is a saturated aliphatic hydrocarbon chain, or an unsaturated aliphatic hydrocarbon chain having at least one double bond, and may have one or more Substituents.

Examples of the Substituents include halogen atoms, hydroxyl, lower (C1-6) alkyl groups, halo lower (C1-6) alkyl groups, hydroxy lower (C1-6) alkyl groups, halo lower (C1-6) alkoxy groups, and others. R³ typically has 11 to 20 carbon atoms. In certain embodiments of the subject invention, R³ has 18 carbon atoms.

SLP are typically produced by yeasts, such as Starmerella spp. yeasts and/or Candida spp. yeasts, e.g., Starmerella (Candida) bombicola, Candida apicola, Candida batistae, Candida floricola, Candida riodocensis, Candida stellate and/or Candida kuoi. SLP have environmental compatibility, high biodegradability, low toxicity, high selectivity and specific activity in a broad range of temperature, pH and salinity conditions. Additionally, in some embodiments, SLP can be advantageous due to their small micelle size, which can help facilitate the movement of the micelle, and compounds enclosed therein, through nanoscale pores and spaces. In certain embodiments, the micelle size of a SLP is less than 100 nm, less than 50 nm, less than 20 nm, less than 15 nm, less than 10 nm, or less than 5 nm.

As used herein, “grinding” refers to the process comprising milling or comminution of particles by, for example, crushing, grinding, cutting, vibrating, or other processes to reduce average particle size and to increase the surface area per unit mass of material. Methods of the invention for grinding particles include the use of rotating ball mills or rotary kilns in which the particles are pulverized. The methods may also involve mills which employ rollers (rotating cylinders) for crushing the particles. For example, the rollers may be used in a paired, nipped configuration, through which the particles are passed and crushed. The rollers may alternatively be used upon a horizontal surface, such as a circular table, on which a bed of particles are crushed as the rollers are rotated over the table surface.

As used herein, “surface energy” or “specific surface energy” represents the quantification of the disruption of intermolecular bonds that occurs when a surface is created, the excess energy at the surface of a material compared to the bulk of the material, or the work required to cut a bulk sample to create two surfaces. The surface energy can be measured by, for example, contact angle measurements.

As used herein, a “grain boundary” is the interface between two grains or crystallites; the grain boundary represents a defect in the crystal structure.

As used herein, “beneficiation” refers to the process by which gangue materials are removed from the product of interest (e.g., element, compound, mineral).

As used herein, “ore” refers to a naturally occurring solid material from which a valuable substance, mineral and/or metal can be profitably extracted. Ores are often mined from ore deposits, which comprise ore minerals containing the valuable substance. “Gangue” minerals are minerals that occur in the deposit but do not contain the valuable substance. Examples of ore deposits include hydrothermal deposits, magmatic deposits, laterite deposits, volcanogenic deposits, metamorphically reworked deposits, carbonatite-alkaline igneous related deposits, placer ore deposits, residual ore deposits, sedimentary deposits, sedimentary hydrothermal deposits and astrobleme-related deposits. Ores, as defined herein, however, can also include ore concentrates or tailings, gold, or even other sources of metal or valuable minerals, including but not limited to, jewelry, electronic scraps, and other scrap materials.

As used herein, the term “particles” includes mined ores, elements, compounds, minerals, or other materials (e.g., gold, silver, mine, lead-zinc, tungsten, zinc, coal, calcite, limestone, aragonite, sea shells, marl, limonite, clay, shale, sand, alumina, taconite, zircon, quartz and bauxite) and also intermediate products produced during beneficiation or manufacturing processes, including, for example, hydratable cement and cement clinker, which is ground, often with gypsum and calcium sulfate, to produce hydratable cement. The present invention not only concerns the grinding of clinker to produce cement and the grinding of cement particles into still finer particles but also the grinding of the raw materials which go into the production of the cement clinker.

As used herein, “leaching” refers to the process by which metal is extracted from ore by aqueous solutions including by, for example, ammonia leaching, alkali leaching, acid leaching, cyanidation (i.e., cyanide leaching), or thiosulfate leaching. As used herein “cyanidation” refers to the process of converting gold in ore to a water-soluble coordination complex using aqueous cyanide, including, for example, sodium cyanide, potassium cyanide, or calcium cyanide.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially of” the recited component(s).

Unless specifically stated or obvious from context, as used herein, the term “or” is understood to be inclusive. Unless specifically stated or obvious from context, as used herein, the terms “a,” “and” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

All references cited herein are hereby incorporated by reference in their entirety.

### Grinding Aid Compositions

In certain embodiments, the subject invention provides compositions comprising components that are derived from microorganisms. In certain embodiments, the composition comprises a microbial biosurfactant. In certain embodiments, the composition comprises one or more biosurfactants, and, optionally, other compounds, such as, for example, water, chemical surfactants, acrylic polymer dispersants, rheology modifiers, polymers, alcohols, glycols, phenols, lignosulfates, ketones, acids, hydrocarbons, fatty acids, fatty acid salts, or any combination thereof.

In certain embodiments, the chemical surfactant of the grinding aid composition is an anionic surfactant, a nonionic surfactant, detergent, wetting agent, emulsifier, foaming agent, and/or dispersant. In certain embodiments, the chemical surfactants include, for example, triethanolamine (TEA), triisopropanolamine, propylene glycol, TEA acetate, and polyglycol phenol ether.

In certain embodiments, the polymers can include amines and salts thereof.

In certain embodiments, the grinding aid composition comprises a microbe-based product comprising a biosurfactant utilized in crude form. The crude form can comprise, in addition to the biosurfactant, fermentation broth in which a biosurfactant-producing microorganism was cultivated, residual microbial cell matter or live or inactive microbial cells, residual nutrients, and/or other microbial growth by-products. The product may be, for example, at least, by weight, 1%, 5%, 10%, 25%, 50%, 75%, or 100% broth. The amount of biomass in the product, by weight, may be, for example, anywhere from 0% to 100% inclusive of all percentages therebetween.

In some embodiments, the biosurfactant is utilized after being extracted from a fermentation broth and, optionally, purified.

The biosurfactant according to the subject invention can be a glycolipid (e.g., sophorolipids, rhamnolipids, cellobiose lipids, mannosylerythritol lipids and trehalose lipids), lipopeptide (e.g., surfactin, iturin, fengycin, arthrofactin and lichenysin), flavolipid, phospholipid (e.g., cardiolipins), fatty acid ester compound, fatty acid ether compound, and/or high molecular weight polymers such as lipoproteins, lipopolysaccharide-protein complexes, and polysaccharide-protein-fatty acid complexes.

In certain specific embodiments, the biosurfactant is a sophorolipid (SLP), including linear SLP, lactonic SLP, acetylated SLP, de-acetylated SLP, salt-form SLP derivatives, esterified SLP derivatives, amino acid-SLP conjugates, and other SLP derivatives or isomers that exist in nature and/or are produced synthetically. In preferred embodiments, the SLP is a linear SLP or a derivatized linear SLP. In certain embodiments, the subject compositions can comprise lactonic and linear SLP, with at least about 50%, about 55%, about 60%, about 65%, about 70%, about 75%, about 80%, about 85%, about 90%, or about 95% of the SLP comprising linear forms, and the remainder comprising, for example, lactonic forms.

In some embodiments, the biosurfactant can be included in the composition at 0.01 to 99.9%, 0.1 to 90%, 0.5 to 80%, 0.75 to 70%, 1.0 to 50%, 1.5 to 25%, or 2.0 to 15% by weight, with respect to the total grinding aid composition.

In another embodiment, a purified biosurfactant may be added in combination with an acceptable carrier, in that the biosurfactant may be presented at concentrations of 0.001 to 50% (v/v), preferably, 0.01 to 20% (v/v), more preferably, 0.02 to 5% (v/v).

In some embodiments, the biosurfactant can be included in the composition at, for example, 0.01 to 100,000 ppm, 0.05 to 10,000 ppm, 0.1 to 1,000 ppm, 0.5 to 750 ppm, 1.0 to 500 ppm, 2.0 to 250 ppm, or 3.0 to 100 ppm, with respect to the amount of liquid being treated.

In certain embodiments, the chemical surfactant of the grinding aid composition is a detergent, wetting agent, emulsifier, foaming agent, and/or dispersant. In some embodiments, the chemical surfactant can be included in the composition at 0.01 to 99.9%, 0.1 to 90%, 0.5 to 80%, 0.75 to 70%, 1.0 to 50%, 1.5 to 25%, or 2.0 to 15% by weight, with respect to the total grinding composition.

The grinding aid composition can further comprise other additives such as, for example, carriers, other microbe-based compositions, additional biosurfactants, enzymes, catalysts, solvents, salts, buffers, chelating agents, acids, emulsifying agents, lubricants, solubility controlling agents, preservatives, stabilizers, ultra-violet light resistant agents, viscosity modifiers, preservatives, tracking agents, and other microbes and other ingredients specific for an intended use.

In certain embodiments, chelating agents can be, but are not limited to, ethylenediaminetetraacetic acid (EDTA), nitrilotriacetic acid (NTA), a phosphonate, succimer (DMSA), diethylenetriaminepentaacetate (DTPA), N-acetyleysteine, n-hydroxyethylethylenediaminetriacetic acid (HEDTA), organic acids with more than one coordination group (e.g., rubeanic acid), STPP (sodiumtripolyphosphate, Na5P3O10), trisodium phosphate (TSP), water, carbohydrates, organic acids with more than one coordination group (e.g., citric acid), lipids, steroids, amino acids or related compounds (e.g., glutathione), peptides, phosphates, nucleotides, tetrapyrrols, ferrioxamines, ionophores, orphenolics, sodium citrate, sodium gluconate, ethylenediamine disuccinic acid (EDDS), iminodisuccinic acid (IDS), L-glutamic acid diacetic Acid (GLDA), GLDA-Na₄, methyl glycindiacetic acid (MGDA), polyaspartic acid (PASA), hemoglobin, chlorophyll, lipophilic β-diketone, and (14,16)-hentriacontanedione, ethylenediamine-N,N′-diglutaric acid (EDDG), ethylenediamine-N,N′-dimalonic acid (EDDM), 3-hydroxy-2,2-iminodisuccinic acid (HIDS), 2-hydroxyethyliminodiacetic acid (HEIDA), pyridine-2,6-dicarboxylic acid (PDA), trimethyl glycine (TMG), Tiron, or any combination thereof.

### Methods of Grinding

In certain embodiments, the subject invention provides a method for grinding particles from various sources, including, for example, mining sites, quarrying sites, and industrial sites.

In certain embodiments, the subject invention provides a method for grinding tailings from mines. The method comprises adding the subject compositions to the tailings. The particles can be more efficiently ground yielding a larger amount of ground particles over a time period, and the particles can achieve a more consistently homogenous size. In preferred embodiments, the tailings are low-grade tailings, in which the tailings comprise less than about 50%, about 40%, about 35%, about 30%, or about 25% of the product of interest (e.g., metal, mineral, compound or element being mined), with the remainder comprising gangue.

In certain embodiments, the mining site can be a coal mine, iron ore mine (e.g., taconite), copper mine, copper-nickel mine, tin mine, nickel mine, gold mine, silver mine, molybdenum mine, aluminum mine (e.g., bauxite mine, kyanite mine), lead-zinc mine, tungsten mine, zirconium mine (e.g., zircon), or zinc mine. The mine can be an underground mine, surface mine, placer mine or in situ mine. In certain embodiments, the quarrying site can extract chalk, clay, cinder, coal, sand, gravel, coquina, diabase, gabbro, granite, gritstone, gypsum, limestone, marble, ores, phosphate rock, quartz, sandstone, slate, travertine, or any combination thereof.

In certain embodiments, ore concentrates or tailings or even other sources of metal or valuable minerals, including but not limited to, jewelry, electronic scraps, and other scrap materials can be ground using the grinding aid compositions and methods. Electronic scraps include, for example, wiring, telephones, batteries, cell phones, computer, watches, appliances, televisions, lamps, toys, medical devices, light bulbs, solar panels, and monitors.

In certain embodiments, the microbe-containing and/or biosurfactant-containing composition can lower the specific surface energy of ore and minerals, and therefore decrease the amount of energy required to create new surfaces during grinding. In such cases, the net decrease in grinding energy required may result either from weakened grain boundary planes due to interparticle penetration of surfactants or from a net decrease in the cohesive force that bonds the particles together (the specific surface energy).

In certain embodiments, the composition can be used during the physical movement of the rock. Examples of physical movement include grinding, tilling, digging, tunneling, excavating, transporting, dumping, crushing, blasting, separating, pelleting, cutting, casting, quarrying, dredging, fracturing, or any combination thereof.

The compositions can be applied to liquids or solids that reside at a range of temperatures and environments, such as, for example, a containment pond, holding tank, belt, tubing, piping, griding ball, tumbler, kiln, silo, mill, roller, or drum.

The grinding aid composition can be applied to a liquid or solid and, optionally, mixed by adding, pouring, or combining.

In certain embodiments, the time period in which the grinding aid composition can be contacted to a liquid or solid is for about 1 second to about 1 year, about 1 minute to about 1 year, about 1 minute to about 6 months, about 1 minute to about 1 month, about 1 minute to about 1 week, about 1 minute to about 48 hours, about 30 minutes to 40 hours, or preferably about 12 hours to 24 hours. In certain embodiments, the methods comprise applying a liquid or solid form of the grinding aid composition to the liquid containing particles for the period of time in which liquid containing particles are being ground or until the size of particles has been reduced to an amount that is determined to be satisfactory, which can be readily determined by one skilled in the art. In certain embodiments, the methods comprise applying a liquid or solid form of the grinding aid composition to the solid for the period of time in which the solid is being ground or until the size of particles has been reduced to an amount that is determined to be satisfactory, which can be readily determined by one skilled in the art. The size of the particles may be considered acceptable depending on the context. For example, the size of particles may be acceptable at a larger diameter at coal mining sites than during the production of Portland cement.

In certain embodiments, the amount of the grinding aid composition applied is about 0.00001 to 15%, about 0.00001 to 10%, about 0.0001 to 5%, about 0.001 to 3%, or 0.02% to about 0.08 vol % based on an amount of liquid that is treated. In certain embodiments, the amount of the grinding aid composition applied is about 0.00001 to 15%, about 0.00001 to 10%, about 0.0001 to 5%, about 0.001 to 3%, or about 0.02% to about 0.08 weight % based on an amount of solid that is treated.

In certain embodiments, the grinding aids are consumed during methods of grinding. Typical consumption rates of grinding aids are about 0.02% to about 0.08%, 0.04% to about 0.08%, or about 0.02% to about 0.03% for cement and about 0.02% to about 0.05% for limestone. In certain embodiments, the grinding aids can be dispensed through a calibrated dosing system.

In certain embodiments, the methods of the subject invention result in at least a 5% increase in the rate of the grinding of solids or liquids containing solids, preferably at least a 10% increase, after one treatment. In certain embodiments, the solid or liquid containing solids can be treated multiple times to further increase the total amount of ground particles and/or further increase the rate of grinding.

In certain embodiments, the grinding aid composition according to the subject invention is effective due to decreasing the specific surface energy of particles in a liquid or solid containing a mineral, ore, element, or other material. For example, in some embodiments, a sophorolipid will form a micelle neutralizing the particles, wherein the micelle is less than 500 μm, less than 100 μm, less than 10 μm, less than 1 μm, less than 100 nm, less than 50 nm, less than 25 nm, less than 15 nm or less than 10 nm in size.

In certain embodiments, the grinding aid compositions can be used in methods of processing ores, ore slurries, or other products obtained via mining.

In certain embodiments, the grinding aid compositions can be used in beneficiation processes, particularly in low-grade ores containing low concentrations of the element or other material, such as, for example, gold or silver. In order to extract the element or compound of interest, it can be necessary to crush and grind the ore and preconcentrate or separate the element or material from the ore by flotation or gravity separation. In certain embodiments, the grinding rate of ore can be accelerated, and the ore processing efficiency can be improved by adding the grinding aid compositions during the beneficiation process.

In certain embodiments, the grinding aid compositions can be used in methods of leaching, such as, for example, gold cyanidation. The process of extraction by leaching includes crushing or grinding or gold ore, leaching (e.g., cyanide leaching), washing and filtering of leaching pulp, extraction of the metal from the leaching solution or pulp, and smelting of finished products. In certain embodiments, the grinding aid compositions can be used in methods of grinding ore, in which the grinding aid compositions increase the rate and/or efficiency of grinding the ore. The grinding of the ore can enhance the leaching efficiency by increasing the surface area of the ore to which the leaching solution (e.g., sodium cyanide) can interact.

In certain embodiments, the subject grinding aid compositions can be used instead of traditional grinding aids or in conjunction with traditional grinding aids. The subject grinding aids can neutralize negative or positive charges on the surface of particles, thereby inhibiting the agglomeration of particles. The grinding aid compositions can weaken grain boundary planes due to interparticle penetration of surfactants or decrease in the cohesive force that bonds the particles together (the specific surface energy).

In certain embodiments, the grinding aid composition can be used in tube mills, rod mills, ball mills, and in autogenous and semi-autogenous grinding that can process on a scale measured in tens, hundreds, or thousands of tons of material. Additionally, the grinding composing can be used in small-scale grinding operations (i.e., less than 10 tons of material), often used of high-value commodities including, for example, gold, or in research laboratories.

In certain embodiments, the grinding aid composition can be used in various industrial methods, including in the manufacturing or processing of cement. During the manufacturing of products, grinding aid compositions can be used primarily in the grinding minerals to obtain a smaller particle, including, for example, a powder. In certain embodiments, calcium sulfate (e.g., gypsum or anhydrite) is added to cement clinker, which is a sintered mixture of limestone and aluminosilicate materials, including, for example clay. In certain embodiments, the mixture is finely ground to form the finished cement powder using grinding aids. In certain embodiments, the grinding process is controlled to obtain a powder with a broad particle size range, in which about 15% by mass comprises particles below 5 μm diameter and about 5% of particles above 45 μm. The measure of fineness usually used is the “specific surface area”, which is the total particle surface area of a unit mass of cement.

Advantageously, in certain embodiments, the grinding aid composition according to the subject invention provides enhanced or increased efficiency of grinding particles with limited negative environmental impacts. Additionally, the methods of the subject invention do not require complicated equipment or high energy consumption, and the production of the grinding aid composition can be performed on site, including, for example, at a mine or at an industrial site. In certain embodiments, the subject grinding aid composition can result in a decreased use of chemical surfactants, synthetic grinding aids, or other potentially harmful chemicals used for grinding.

### Production of Microbe-Based Products

In certain embodiments, the subject invention provides methods for cultivation of microorganisms and production of microbial metabolites and/or other by-products of microbial growth. The subject invention further utilizes cultivation processes that are suitable for cultivation of microorganisms and production of microbial metabolites on a desired scale. These cultivation processes include, but are not limited to, submerged cultivation/fermentation, solid state fermentation (SSF), and modifications, hybrids and/or combinations thereof.

The microorganisms can be, for example, bacteria, yeast and/or fungi. These microorganisms may be natural, or genetically modified microorganisms. For example, the microorganisms may be transformed with specific genes to exhibit specific characteristics. The microorganisms may also be mutants of a desired strain. As used herein, “mutant” means a strain, genetic variant or subtype of a reference microorganism, wherein the mutant has one or more genetic variations (e.g., a point mutation, missense mutation, nonsense mutation, deletion, duplication, frameshift mutation or repeat expansion) as compared to the reference microorganism. Procedures for making mutants are well known in the microbiological art. For example, UV mutagenesis and nitrosoguanidine are used extensively toward this end.

In certain embodiments, the microbes are capable of producing amphiphilic molecules, enzymes, proteins and/or biopolymers. Microbial biosurfactants, in particular, are produced by a variety of microorganisms such as bacteria, fungi, and yeasts, including, for example, Agrobacterium spp. (e.g., A. radiobacter); Arthrobacter spp.; Aspergillus spp.; Aureobasidium spp. (e.g., A. pullulans); Azotobacter (e.g., A. vinelandii, A. chroococcum); Azospirillum spp. (e.g., A. brasiliensis); Bacillus spp. (e.g., B. subtilis, B. amyloliquefaciens, B. pumillus, B. cereus, B. licheniformis, B. firmus, B. laterosporus, B. megaterium); Blakeslea; Candida spp. (e.g., C. albicans, C. rugosa, C. tropicalis, C. lipolytica, C. torulopsis); Clostridium (e.g., C. butyricum, C. tyrobutyricum, C. acetobutyricum, and C. beijerinckii); Campylobacter spp.; Cornybacterium spp.; Cryptococcus spp.; Debaryomyces spp. (e.g., D. hansenii); Entomophthora spp.; Flavobacterium spp.; Gordonia spp.; Hansenula spp.; Hanseniaspora spp. (e.g., H. uvarum); Issatchenkia spp; Kluyveromyces spp.; Meyerozyma spp. (e.g., M. guilliermondii); Mortierella spp.; Mycorrhiza spp.; Mycobacterium spp.; Nocardia spp.; Pichia spp. (e.g., P. anomala, P. guilliermondii, P. occidentalis, P. kudriavzevii); Phycomyces spp.; Phythium spp.; Pseudomonas spp. (e.g., P. aeruginosa, P. chlororaphis, P. putida, P. florescens, P. fragi, P. syringae); Pseudozyma spp. (e.g., P. aphidis); Ralslonia spp. (e.g., R. eulropha); Rhodococcus spp. (e.g., R. erythropolis); Rhodospirillum spp. (e.g., R. rubrum); Rhizobium spp.; Rhizopus spp.; Saccharomyces spp. (e.g., S. cerevisiae, S. boulardii sequela, S. torula); Sphingomonas spp. (e.g., S. paucimobilis); Starmerella spp. (e.g., S. bombicola); Thraustochytrium spp.; Torulopsis spp.; Ustilago spp. (e.g., U. maydis); Wickerhamomyces spp. (e.g., W. anomalus); Williopsis spp.; and/or Zygosaccharomyces spp. (e.g., Z. bailii).

In preferred embodiments, microorganism is a Starmerella spp. yeast and/or Candida spp. yeast, e.g., Starmerella (Candida) bombicola, Candida apicola, Candida batistae, Candida floricola, Candida riodocensis, Candida stellate and/or Candida kuoi. In a specific embodiment, the microorganism is Starmerella bombicola, e.g., strain ATCC 22214.

As used herein “fermentation” refers to cultivation or growth of cells under controlled conditions. The growth could be aerobic or anaerobic. In preferred embodiments, the microorganisms are grown using SSF and/or modified versions thereof.

In one embodiment, the subject invention provides materials and methods for the production of biomass (e.g., viable cellular material), extracellular metabolites (e.g., small molecules and excreted proteins), residual nutrients and/or intracellular components (e.g., enzymes and other proteins).

The microbe growth vessel used according to the subject invention can be any fermenter or cultivation reactor for industrial use. In one embodiment, the vessel may have functional controls/sensors or may be connected to functional controls/sensors to measure important factors in the cultivation process, such as pH, oxygen, pressure, temperature, humidity, microbial density and/or metabolite concentration.

In a further embodiment, the vessel may also be able to monitor the growth of microorganisms inside the vessel (e.g., measurement of cell number and growth phases). Alternatively, a daily sample may be taken from the vessel and subjected to enumeration by techniques known in the art, such as dilution plating technique. Dilution plating is a simple technique used to estimate the number of organisms in a sample. The technique can also provide an index by which different environments or treatments can be compared.

In one embodiment, the method includes supplementing the cultivation with a nitrogen source. The nitrogen source can be, for example, potassium nitrate, ammonium nitrate ammonium sulfate, ammonium phosphate, ammonia, urea, and/or ammonium chloride. These nitrogen sources may be used independently or in a combination of two or more.

The method can provide oxygenation to the growing culture. One embodiment utilizes slow motion of air to remove low-oxygen containing air and introduce oxygenated air. In the case of submerged fermentation, the oxygenated air may be ambient air supplemented daily through mechanisms including impellers for mechanical agitation of liquid, and air spargers for supplying bubbles of gas to liquid for dissolution of oxygen into the liquid.

The method can further comprise supplementing the cultivation with a carbon source. The carbon source is typically a carbohydrate, such as glucose, sucrose, lactose, fructose, trehalose, mannose, mannitol, and/or maltose; organic acids such as acetic acid, fumaric acid, citric acid, propionic acid, malic acid, malonic acid, and/or pyruvic acid; alcohols such as ethanol, propanol, butanol, pentanol, hexanol, isobutanol, and/or glycerol; fats and oils such as soybean oil, canola oil, rice bran oil, olive oil, corn oil, sesame oil, and/or linseed oil; etc. These carbon sources may be used independently or in a combination of two or more.

In one embodiment, growth factors and trace nutrients for microorganisms are included in the medium. This is particularly preferred when growing microbes that are incapable of producing all of the vitamins they require. Inorganic nutrients, including trace elements such as iron, zinc, copper, manganese, molybdenum and/or cobalt may also be included in the medium. Furthermore, sources of vitamins, essential amino acids, and microelements can be included, for example, in the form of flours or meals, such as corn flour, or in the form of extracts, such as yeast extract, potato extract, beef extract, soybean extract, banana peel extract, and the like, or in purified forms. Amino acids such as, for example, those useful for biosynthesis of proteins, can also be included.

In one embodiment, inorganic salts may also be included. Usable inorganic salts can be potassium dihydrogen phosphate, dipotassium hydrogen phosphate, disodium hydrogen phosphate, magnesium sulfate, magnesium chloride, iron sulfate, iron chloride, manganese sulfate, manganese chloride, zinc sulfate, lead chloride, copper sulfate, calcium chloride, sodium chloride, calcium carbonate, and/or sodium carbonate. These inorganic salts may be used independently or in a combination of two or more.

In some embodiments, the method for cultivation may further comprise adding additional acids and/or antimicrobials in the medium before, and/or during the cultivation process. Antimicrobial agents or antibiotics are used for protecting the culture against contamination.

Additionally, antifoaming agents may also be added to prevent the formation and/or accumulation of foam during submerged cultivation.

The pH of the mixture should be suitable for the microorganism of interest. Buffers, and pH regulators, such as carbonates and phosphates, may be used to stabilize pH near a preferred value. When metal ions are present in high concentrations, use of a chelating agent in the medium may be necessary.

The microbes can be grown in planktonic form or as biofilm. In the case of biofilm, the vessel may have within it a substrate upon which the microbes can be grown in a biofilm state. The system may also have, for example, the capacity to apply stimuli (such as shear stress) that encourages and/or improves the biofilm growth characteristics.

In one embodiment, the method for cultivation of microorganisms is carried out at about 5° to about 100° C., preferably, 15 to 60° C., more preferably, 25 to 50° C. In a further embodiment, the cultivation may be carried out continuously at a constant temperature. In another embodiment, the cultivation may be subject to changing temperatures.

In one embodiment, the equipment used in the method and cultivation process is sterile. The cultivation equipment such as the reactor/vessel may be separated from, but connected to, a sterilizing unit, e.g., an autoclave. The cultivation equipment may also have a sterilizing unit that sterilizes in situ before starting the inoculation. Air can be sterilized by methods know in the art. For example, the ambient air can pass through at least one filter before being introduced into the vessel. In other embodiments, the medium may be pasteurized or, optionally, no heat at all added, where the use of low water activity and low pH may be exploited to control undesirable bacterial growth.

In one embodiment, the subject invention further provides a method for producing microbial metabolites such as, for example, biosurfactants, enzymes, proteins, ethanol, lactic acid, beta-glucan, peptides, metabolic intermediates, polyunsaturated fatty acid, and lipids, by cultivating a microbe strain of the subject invention under conditions appropriate for growth and metabolite production; and, optionally, purifying the metabolite. The metabolite content produced by the method can be, for example, at least 20%, 30%, 40%, 50%, 60%, 70%, 80%, or 90%.

The microbial growth by-product produced by microorganisms of interest may be retained in the microorganisms or secreted into the growth medium. The medium may contain compounds that stabilize the activity of microbial growth by-product.

The biomass content of the fermentation medium may be, for example, from 5 g/l to 180 g/l or more, or from 10 g/l to 150 g/l.

The cell concentration may be, for example, at least 1×10⁶ to 1×10¹², 1×10⁷ to 1×10¹¹, 1×10⁸ to 1×10¹⁰, or 1×10⁹ CFU/ml.

The method and equipment for cultivation of microorganisms and production of the microbial by-products can be performed in a batch, a quasi-continuous process, or a continuous process.

In one embodiment, all of the microbial cultivation composition is removed upon the completion of the cultivation (e.g., upon, for example, achieving a desired cell density, or density of a specified metabolite). In this batch procedure, an entirely new batch is initiated upon harvesting of the first batch.

In another embodiment, only a portion of the fermentation product is removed at any one time. In this embodiment, biomass with viable cells, spores, conidia, hyphae and/or mycelia remains in the vessel as an inoculant for a new cultivation batch. The composition that is removed can be a cell-free medium or contain cells, spores, or other reproductive propagules, and/or a combination of thereof. In this manner, a quasi-continuous system is created.

Advantageously, the method does not require complicated equipment or high energy consumption. The microorganisms of interest can be cultivated at small or large scale on site and utilized, even being still-mixed with their media.

In certain embodiments, the subject invention provides a “microbe-based composition,” meaning a composition that comprises components that were produced as the result of the growth of microorganisms or other cell cultures. Thus, the microbe-based composition may comprise the microbes themselves and/or by-products of microbial growth. The microbes may be in a vegetative state, in spore form, in mycelial form, in any other form of propagule, or a mixture of these. The microbes may be planktonic or in a biofilm form, or a mixture of both. The by-products of growth may be, for example, metabolites, cell membrane components, expressed proteins, and/or other cellular components. The microbes may be intact or lysed. The microbes may be present in or removed from the composition. The microbes can be present, with broth in which they were grown, in the microbe-based composition. The cells may be present at, for example, a concentration of at least 1×10³, 1×10⁴, 1×10⁵, 1×10⁶, 1×10⁷, 1×10⁸, 1×10⁹, 1×10¹⁰, 1×10¹¹, 1×10¹², 1×10¹³ or more CFU per milliliter of the composition.

The subject invention further provides “microbe-based products,” which are products that are to be applied in practice to achieve a desired result. The microbe-based product can be simply a microbe-based composition harvested from the microbe cultivation process. Alternatively, the microbe-based product may comprise further ingredients that have been added. These additional ingredients can include, for example, stabilizers, acids, buffers, carriers, such as water, salt solutions, or any other appropriate carrier, added nutrients to support further microbial growth, non-nutrient growth enhancers, and/or agents that facilitate tracking of the microbes and/or the composition in the environment to which it is applied. The microbe-based product may also comprise mixtures of microbe-based compositions. The microbe-based product may also comprise one or more components of a microbe-based composition that have been processed in some way such as, but not limited to, filtering, centrifugation, lysing, drying, purification and the like.

One microbe-based product of the subject invention is simply the fermentation medium containing the microorganisms and/or the microbial metabolites produced by the microorganisms and/or any residual nutrients. The product of fermentation may be used directly without extraction or purification. If desired, extraction and purification can be easily achieved using standard extraction and/or purification methods or techniques described in the literature.

The microorganisms in the microbe-based products may be in an active or inactive form, or in the form of vegetative cells, reproductive spores, conidia, mycelia, hyphae, or any other form of microbial propagule. The microbe-based products may also contain a combination of any of these forms of a microorganism.

In one embodiment, different strains of microbe are grown separately and then mixed together to produce the microbe-based product. The microbes can, optionally, be blended with the medium in which they are grown and dried prior to mixing.

The microbe-based products may be used without further stabilization, preservation, and storage. Advantageously, direct usage of these microbe-based products preserves a high viability of the microorganisms, reduces the possibility of contamination from foreign agents and undesirable microorganisms, and maintains the activity of the by-products of microbial growth.

Upon harvesting the microbe-based composition from the growth vessels, further components can be added as the harvested product is placed into containers or otherwise transported for use. The additives can be, for example, buffers, carriers, other microbe-based compositions produced at the same or different facility, viscosity modifiers, preservatives, nutrients for microbe growth, surfactants, emulsifying agents, lubricants, solubility controlling agents, tracking agents, solvents, biocides, antibiotics, pH adjusting agents, chelators, stabilizers, ultra-violet light resistant agents, other microbes and other suitable additives that are customarily used for such preparations.

Optionally, the product can be stored prior to use. The storage time is preferably short. Thus, the storage time may be less than 60 days, 45 days, 30 days, 20 days, 15 days, 10 days, 7 days, 5 days, 3 days, 2 days, 1 day, or 12 hours. In a preferred embodiment, if live cells are present in the product, the product is stored at a cool temperature such as, for example, less than 20° C., 15° C., 10° C., or 5° C. On the other hand, a biosurfactant composition can typically be stored at ambient temperatures.

1. A method of grinding, the method comprising contacting a grinding aid composition comprising a glycolipid biosurfactant to a liquid containing particles or to a solid particle to yield a ground particle.

2. The method of claim 1, further comprising removing said ground particle from the liquid.

3. The method of claim 1, wherein the solid comprises cement clinker.

4. The method of claim 1, wherein the solid is a metal ore.

5. The method of claim 1, wherein the liquid is a slurry of ore.

6. The method of claim 4, wherein the metal ore is from a coal mine, iron ore mine, copper mine, copper-nickel mine, tin mine, nickel mine, gold mine, silver mine, molybdenum mine, aluminum mine, lead-zinc mine, tungsten mine, or zinc mine.

7. The method of claim 5, wherein the slurry of ore is from a coal mine, iron ore mine, copper mine, copper-nickel mine, tin mine, nickel mine, gold mine, silver mine, molybdenum mine, aluminum mine, lead-zinc mine, tungsten mine, or zinc mine.

8. The method of claim 1, wherein the solid is chalk, clay, cinder, coal, sand, gravel, coquina, diabase, gabbro, granite, gritstone, gypsum, limestone, marble, ores, phosphate rock, quartz, sandstone, slate, travertine, or any combination thereof.

9. The method of claim 1, wherein the grinding aid composition further comprises a chemical surfactant, acrylic polymer dispersant, rheology modifier, polymer, alcohol, glycol, phenol, lignosulfate, ketone, acid, hydrocarbon, fatty acid, fatty acid salt, or any combination thereof.

10-11. (canceled)

12. The method of claim 1, wherein the concentration of the grinding aid composition contacted to the solid is about 0.001% to about 1% by weight or to the liquid is about 0.001% to about 1% by volume.

13. The method of claim 1, wherein the glycolipid biosurfactant is a sophorolipid and/or a yeast culture comprising a sophorolipid.

14. The method of claim 13, wherein the yeast culture is a Starmerella sp. and/or a Candida sp. yeast.

15. The method of claim 13, wherein the yeast is in a vegetative state.

16. The method of claim 13, wherein the yeast is in a spore form.

17. The method of claim 1, wherein the grinding of particles comprises one or a combination of the following:
a) decreasing the specific surface energy of the particles in the liquid or the solid particle;
b) decreasing the cohesive force that binds the particles in the liquid together or the solid particle together;
c) weakening the grain boundary planes of the particles in the liquid or the solid particle; or
d) neutralizing the particles in the liquid or the solid particle.

18. A grinding aid composition comprising a sophorolipid and/or a yeast culture comprising a sophorolipid and one or more traditional grinding aid components.

19. The composition of claim 18, wherein the yeast culture is a Starmerella sp. and/or a Candida sp. yeast.

20. (canceled)

21. The composition of claim 18, wherein the yeast is in a spore form.

22. The composition of claim 18, wherein the traditional grinding aid components are selected from one or a combination of the following:
a) chemical surfactant;
b) polymer;
c) water;
d) acrylic polymer dispersant;
e) rheology modifier;
f) alcohol;
g) glycol;
h) phenol;
i) lignosulfate;
j) ketone;
k) acid;
l) hydrocarbon;
m) fatty acid; or
n) fatty acid salt.

23. The composition of claim 22, wherein the chemical surfactant is triethanolamine (TEA), triisopropanolamine, propylene glycol, TEA acetate, polyglycol phenol ether, or any combination thereof.
